(19) United States
(12) Patent Application Publication
(10) Pub. No.: US 2022/0409601 A1
(43) Pub. Date: Dec. 29, 2022

(54) COMBINATION CANNABINOID-NAD+ PRECURSOR FORMULATION FOR TREATMENT OF INFLAMMATION AND METHODS RELATED THERETO

(71) Applicant: Canole LLC, Shreveport, LA (US)

(72) Inventors: David Bradley Schmidt, Shreveport, LA (US); Alana L. Gray, Keithville, LA (US); David T. Coleman, Keithville, LA (US)

(73) Assignee: CANOLE LLC, Shreveport, LA (US)

(21) Appl. No.: 17/778,821

(22) PCT Filed: Nov. 20, 2020
(86) PCT No.: PCT/US20/61648

§ 371 Date: May 20, 2022

Related U.S. Application Data
(60) Provisional application No. 62/938,099, filed on Nov. 20, 2019.

(51) Int. Cl. A61K 31/455; A61K 31/05; A61P 29/00; A61K 9/00

(52) U.S. Cl. CPC A61K 31/455; A61K 31/05; A61P 29/00; A61K 9/0053; A61K 9/0014; A61K 9/007

(57) ABSTRACT

The present invention describes cannabinoid formulations that combine cannabinoids, such as CBD, with other active agents within the NAD+ precursor class of compounds which, in combination, provide synergistic anti-inflammatory effects. These preparations are capable of increasing anti-inflammatory effects when compared to each individual compound, and are further capable of delivery through a variety of administration routes.

### CROSS-REFERENCE TO RELATED APPLICATIONS

This application claims the benefit of U.S. Provisional Patent Application Ser. No. 62/938,099 filed on Nov. 20, 2019.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### FIELD

The present invention relates in general to the field of drug delivery formulations. In particular, the present invention provides for a combination cannabinoid formulation for treatment of inflammation when delivered to a patient.

### BACKGROUND

Cannabis is an annual, primarily dioecious, flowering herb. The genera Cannabis is considered to be monospecific (Cannabis sativa L.) which is divided into several subspecies (C. sativa subsp. sativa, C. sativa subsp. indica, C. sativa subsp. ruderalis, C. sativa subsp. spontanea, C. sativa subsp. kafiristanca). However, the chemical and morphological distinctions by which cannabis has been split into these subspecies are often not readily discernible, appear to be environmentally modifiable, and vary in a continuous fashion. For most purposes, it will suffice to apply the name Cannabis sativa to all cannabis plants encountered.

Cannabinoids are chemical compounds found in the Cannabis plant that interact with receptors in the brain and body to create various effects. Cannabis contains over 400 compounds including over 100 cannabinoids, which are aryl-substituted meroterpenes unique to the plant genus cannabis. The pharmacology of most of the cannabinoids is largely unknown but the most potent psychoactive agent, Δ⁹-tetrahydrocannabinol (Δ⁹-THC, or THC), has been isolated, synthesized and much studied due to its abundance and psychoactive attributes. Other plant-based cannabinoids include Δ⁹-tetrahydrocannabinolic acid, Δ⁸-THC, cannabigerol, cannabidiolic acid, and cannabidiol (CBD). These and other cannabinoids have additive, synergistic or antagonistic effects with THC and may modify its actions when cannabis products are consumed.

Certain cannabinoids have little to no psychoactive effects as compared to THC. In particular, CBD has received significant focus as a wellness option. Benefits of CBD have been described in the literature to include anti-inflammatory effects. However, despite efforts to create effective anti-inflammatory cannabinoid formulations, there remains a need in the art for cannabinoid formulations that have increased efficacy and which are conducive to use with traditional drug delivery methods.

### SUMMARY OF THE DISCLOSURE

It is therefore an object of the present invention to provide for cannabinoid formulations that combine CBD with other active agents within the oxidized nicotinamide adenine dinucleotide (NAD+) precursor class of compounds which, in combination, provide synergistic anti-inflammatory effects.

In one aspect of the present invention, a composition is provided for treatment of inflammatory conditions, said composition comprising: at least one cannabinoid; and at least one NAD+ precursor; wherein said composition is capable of having anti-inflammatory effects when administered to a patient. In one aspect the ratio of the at least one cannabinoid and the at least one NAD+ precursor is between 200:1 and 1:200. In another aspect the ratio of the at least one cannabinoid and the at least one NAD+ precursor is between 150:1 and 1:150. In yet another aspect, the at least one cannabinoid and the at least one NAD+ precursor is 1:100 and 100:1 In a preferred embodiment of the present invention each dosage formulation provides 0.1 to 100 milligrams of the cannabinoid per dosage.

It is an object of the present invention to provide a composition that is suitable for oral administration, including buccal or sublingual administration. In another aspect the composition is suitable for topical administration. In another aspect, the composition is suitable for mucosal administration. In yet another aspect, said composition is suitable for pulmonary administration. The composition is also suitable for subcutaneous, intravenous, intraperitoneal, suppository or intramuscular administration.

In one aspect of the present invention, the composition is a formulation selected from the group consisting of: a tablet, capsule, spray, drop, solution, suspension, gel, ointment, lotion, cream, powder, transdermal patch, tampon, or a sponge.

It is an object of the present invention that the at least one NAD+ precursor comprises nicotinic acid. In another aspect, the at least one NAD+ precursor is selected from a group consisting of: tryptophan, nicotinic acid, nicotinamide, nicotinamide riboside, nicotinamide ribose, nicotinamide mononucleotide, and combinations thereof. In another aspect rather than an NAD+ precursor one could supply NAD itself along with the cannabinoid.

It is another object of the present invention that the cannabinoid is selected from the group consisting of: cannabidiol (CBD), cannabidivarol (CBDV), cannabinol (CBN), cannabigerol (CBG), cannabivarol (CBV), cannabicyclol (CBL), tetrahydrocannabinol (THC), tetrahydrocannabinol-C4, (THC-C4), tetrahydrocannabivarin (THCV), 11-Hydroxy-Δ9-tetrahydrocannabinol, (11-OH-THC), 11-nor-9-Carboxy-Δ9-tetrahydrocannabinol, and combinations thereof. The at least one cannabinoid is preferably provided in an amount of from 0.1 to 100 milligrams in each dosage of a formulation according to the present invention.

It is another object of the present invention to provide a method of treating inflammation in an animal comprising administering to an animal in need thereof a therapeutically effective amount of a composition comprising at least one cannabinoid; and at least one NAD+ precursor.

It is another object of the present invention to provide a method for preparing a combination formulation having anti-inflammatory properties, the method comprising the steps of: providing at least one cannabinoid; providing at least one NAD+ precursor; combining the at least one cannabinoid with the at least one NAD+ precursor to form a combination formulation; wherein said combination formulation is capable of reducing inflammation in an animal administered said combination formulation.

### BRIEF DESCRIPTION OF THE DRAWINGS

The present invention is further described in the detailed description which follows, in reference to the noted plurality of drawings by way of non-limiting examples of exemplary embodiments, in which like reference numerals represent similar parts throughout the several views of the drawings, and wherein:

FIG. 1. depicts a chart showing the synergistic effect of combination therapies in relation to non-combination therapies on expression of the pro-imflammatory glycoprotein granulocyte-macrophage colony-stimulating factor (GM-CSF) by chondrocytes, the data is picograms per milliliter of GM-CSF.

FIG. 2A depicts a chart showing low concentrations of individual and combination therapies and the corresponding effect on GM-CSF expression in chondrocytes, data is picograms per milliliter.

FIG. 2B depicts a chart showing moderate concentrations of individual and combination therapies and the corresponding effect on GM-CSF expression in chondrocytes, data is picograms per milliliter.

FIG. 2C depicts a chart showing high concentrations of individual and combination therapies and the corresponding effect on GM-CSF expression in chondrocytes, data is picograms per milliliter.

FIG. 3 depicts a chart showing interleukin 4 (IL-4) expression as a result of varying concentrations of individual and combination therapies in chondrocytes, data is picograms per milliliter.

FIG. 4 depicts a chart showing interleukin 13 (IL-13) expression as a result of varying concentrations of individual and combination therapies in chondrocytes, data is picograms per milliliter.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

While the making and using of various embodiments of the present invention are discussed in detail below, it should be appreciated that the present invention provides many applicable inventive concepts that can be embodied in a wide variety of specific contexts, goods, or services. The specific embodiments discussed herein are merely illustrative of specific ways to make and use the disclosure and do not delimit the scope of the disclosure.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the disclosure as set forth in the appended claims.

Specific details are given in the following description to provide a thorough understanding of the embodiments. However, it will be understood by one of ordinary skill in the art that the embodiments may be practiced without these specific details. For example, systems, processes, and other elements in the instant disclosure may be shown as components in block diagram form in order not to obscure the embodiments in unnecessary detail. In other instances, well-known processes, structures, and techniques may be shown without unnecessary detail in order to avoid obscuring the embodiments. Further, like reference numbers and designations in the various drawings indicated like elements.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context

Also, it is noted that individual embodiments may be described as a process which is depicted as a flowchart, a flow diagram, or a block diagram. Although a flowchart may describe the operations as a sequential process, many of the operations can be performed in parallel or concurrently. In addition, the order of the operations may be re-arranged.

In one embodiment of the present invention, a combination treatment of a formulation comprising at least one cannabinoid, such as CBD, and one or more NAD+ precursors, such as niacin or nicotinamide ribose, the combination of the two ingredients capable of decreasing the secretion of inflammatory cytokines by chondrocytes.

Cannabinoids are chemical compounds found in the cannabis plant that interact with receptors in the brain and body to create various effects. Cannabis contains over 400 compounds including over 100 cannabinoids, which are aryl-substituted meroterpenes unique to the plant genus cannabis. The pharmacology of most of the cannabinoids is largely unknown but the most potent psychoactive agent, Δ⁹-tetrahydrocannabinol (Δ⁹-THC, or THC), has been isolated, synthesized and much studied due to its abundance and psychoactive attributes. Other plant-based cannabinoids include Δ⁹-tetrahydrocannabinolic acid, Δ⁸-THC, cannabigerol, cannabidiolic acid, and cannabidiol (CBD). These and other cannabinoids have additive, synergistic or antagonistic effects with THC and may modify its actions when cannabis products are consumed. For purposes of the present invention, the cannabinoid “CBD” is utilized, but is intended to be non-limiting, and can include all cannabinoids including combinations thereof.

NAD+ precursors are a term utilized in the present invention primarily refer to niacin and nicotinamide ribose (NR). Niacin, also known as nicotinic acid, is an organic compound and a form of vitamin B3, an essential human nutrient. It has the formula C₆H₅NO₂ and belongs to the group of the pyridinecarboxylic acid. As the precursor for NAD and nicotinamide adenine dinucleotide phosphate (NADP), niacin is also involved in DNA repair. In 2016 it was the 277th most prescribed medication in the United States with more than a million prescriptions. It has the European food additive series E number of E375.

To date, little research has been done around the medicinal uses of cannabinoids because the federal government currently classifies it as a Schedule I substance, which makes researching the plant extremely difficult. However, there is increasing evidence that cannabinoids, particularly cannabidiol (CBD) have beneficial uses, such as pain management, multiple sclerosis, epilepsy, Parkinson's disease, and post traumatic stress disorder (PTSD). Cannabinoids themselves are typically not strong enough for severe pain management from broken bones or post-surgical pain but is effective for the management of chronic pain, and they are increasingly considered safer and less addictive than opiates and can also be taken as an alternative to nonsteroidal anti-inflammatory drugs (NSAIDs), such as Advil or Aleve.

Currently, there are few effective options to treating nerve pain other than Neurontin, Lyrica or highly sedating opiates. Patients have claimed certain cannabinoids allow them to resume the activities of daily living while not feeling overwhelmed by the side effects of powerful pharmaceutical drugs.

The FDA recently approved a drug called Epidiolex (derived from CBD) as a method to treat people with severe seizures. Some people show a large drop in the frequency of their seizures while taking the drug. Additionally, cannabinoids are known as an effective muscle relaxant, and many people with Parkinson's disease are convinced that it significantly lessens their tremors.

Other applications of cannabinoids include treatment of anorexia, nausea and weight loss. Certain cannabinoids, such as THC, reache the area of the brain that affects appetite and subsequently stimulate eating.

Research has shown that cannabinoids, such as CBD, are able to modulate the immune system and reduce convulsion and inflammatory pain in some animal studies by interacting with the endocannabinoid system, notably the CB1 and CB2 receptors, as well as other receptors, such as the TRPV1, glycine receptor and the like.

CBD has also been shown to reduce inflammatory pain in animal models, but again, not by interacting directly with the body's cannabinoid receptors. Rather, CBD appears to block inflammatory pain by interacting with another protein, the glycine receptor, which plays a critical role in transmitting pain signals from the body, through the spinal cord, and into the brain where pain is actually perceived.

CBD also acts on inflammation by decreasing oxidative stress in the body. Oxidative stress occurs when there is a disturbance between the production of free radicals and antioxidant defenses, resulting in inflammation or tissue damage. CBD possesses antioxidant properties, and has been shown to reduce oxidative stress and inflammation in the body following a potent chemotherapy treatment.

While multiple pathways are mediated by CBD's activity, it is an preferred embodiment of the present invention, that inflammation is itself mediated by chondrocytes. The chondrocyte is the only specialized cell type present in articular cartilage. They produce cytokines, growth factors, and extracellular matrix structural proteins to support and repair cartilage. Human chondrocytes in mature articular cartilage are loosely packed, post-mitotic and terminally differentiated cells making them sensitive to damage resulting in long-term consequences. Each chondrocyte is responsible for the turnover of extracellular matrix in its immediate vicinity. Pro-inflammatory cytokines can be secreted by chondrocytes resulting in feedback that modulates the degradation and synthesis of matrix proteins that make up the cartilage. The development, maintenance, and repair of the extracellular matrix by chondrocytes dictates the health of joints. Dysregulated chondrocyte function leads to chronic pain and inflammation, ultimately leading to arthritis and permanent joint damage potentially requiring surgical repair. Superficial joints like the knees, ankles, feet, elbows, and hands are particularly receptive to topical penetrating medications to alleviate dysregulated inflammation and pain.

In one embodiment, the present invention provides a combination treatment of a formulation comprising at least one cannabinoid, such as CBD, and one or more NAD+ precursors, such as niacin or nicotinamide ribose, the combination of the two ingredients capable of decreasing the secretion of inflammatory cytokines by chondrocytes. As known to one of skill in the art cytokines are small proteins of 5 to 20 Kilo-Daltons that are involved in cell signaling. The typical cytokines include: chemokines, interferons, interleukins, lymphokines and tumor necrosis factors. In the present disclosure the cytokines focused on are interleukins and GM-CSF. Chondrocytes are the only cell type found in the lacunae of cartilage. They are responsible for synthesis of the collagen, proteoglycans and elastin fibers that make up cartilage.

In one embodiment, the composition comprises an aqueous solution and one or more pharmaceutically acceptable excipients, additives, carriers or adjuvants. In another embodiment, the composition further comprises one or more excipients, carriers, additives, adjuvants, or binders in a tablet or capsule.

In another embodiment, the present invention may be in liquid, solid or semisolid dosage forms, including, but not limited to, emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

In another embodiment the composition is administered via an oral, intraperitoneal, intravascular, peripheral circulation, subcutaneous, intraorbital, ophthalmic, intraspinal, intracisternal, topical, infusion, implant, aerosol, inhalation, scarification, intracapsular, intramuscular, intranasal, buccal, sublingual, transdermal, pulmonary, rectal, or vaginal route. The composition is formulated in a dosage form selected from the group consisting of liquid, solid, gas, oral, pill, tablet, capsule, caplet, buccal, sub-lingual, orally-disintegrating, thin film, liquid solution, suspension, powder or liquid or solid crystals, pastes, inhalational, aerosol, inhaler, nebulizer, smoking, vaporizer, spray, syrup, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof.

In another embodiment, the composition further comprises a hydrophobic component selected from the group consisting of: cannabis oil, borage oil, coconut oil, medium chain triglyceride (MCT) oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof.

The term “pharmaceutically acceptable carrier”, “excipient”, or “vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, pectin, dextrose, water, glycerol, ethanol, and combinations thereof. The use of such media and agents for an active substance is well known in the art.

The pharmaceutically acceptable carrier may be selected from the group consisting of water, saline, cyclodextrin, glycerol, or combinations thereof. In one aspect, the pharmaceutically acceptable carrier is β-cyclodextrin. In another aspect, the pharmaceutically acceptable carrier is: α-cyclodextrin, β-cyclodextrin, γ-cyclodextrin, methylated β-cyclodextrin, hydroxypropyl- and hydroxyethyl-cyclodextrin (di)glucosyl- or (di)maltosyl-cyclodextrins, carboxymethyl-cyclodextrins, or sulfobutylether-β-cyclodextrin.

In another embodiment, preservatives may be used, referring to one or more of benzalkonium, benzalkonium chloride, potassium sorbate, benzyl alcohol, thimerosal (merthiolate), edetate disodium monobasic sodium phosphate, providone, di-basic sodium phosphate, disodium ETA, potassium phosphate monobasic, iodine, phenylcarbinol, sodium silicoaluminate, and the like. Indeed, other carriers, preservatives, buffers, moisturizers, or volatile oils or fragrances may be used in the composition of the present invention.

The composition of the present invention may further comprise a buffer system for increased stability/adjusting pH to reduce irritation. A preferred buffer system includes sodium chloride, calcium chloride, disodium hydrogen phosphate and calcium hydrogen phosphate, in a concentration sufficient to maintain the pH of the composition at a value inclusive of pH 5.5 to pH 8.5.

### Example—Cytokine Expression Changes

A clonal derivative of ATCC-certified normal chondrocytes was used for a study to determine cytokine expression. The parent line was originally derived from costal cartilage of adolescent female. The cell line used is C-28/I2 accession number CVCL 0187. The cells were immortalized via retroviral vector-mediated SV40 Large T antigen expression. Cells were confirmed negative for mycoplasma contamination and genotyped by STR analysis to verify cell identity. This cell line is widely used as a model for studying normal and pathological cartilage repair mechanisms and inflammation.

The cells were cultured at 37° C. with 5% CO₂, DMEM/F12 media supplemented with 10% heat-inactivated fetal bovine serum. Antimicrobials were not added to the media. Cells were maintained between 40 and 80% confluency to avoid terminal differentiation. Cells used for the experiments had been passaged no more than ten times prior to each experiment.

Stock solutions of cannabidiol (CBD, mol. wt 314.47, isolate), niacin/nicotinic acid (Sigma, ≥99.5% HPLC, mol. wt: 123.11), and nicotinamide ribose (Sigma, ≥99.5% HPLC, mol wt: 255.25) and oleocanthal (Oleolive, ≥98% HPLC, mol wt: 304.34) were made up in DMSO to final concentrations of 50 millimolar (mM), 150 mM, 150 mM, and 50 mM respectively. Stock solutions were aliquoted to minimize freeze-thaw cycles and stored at −20° C. Recombinant Human IL-10 and IL-6 (PeproTech, ≥98% HPLC) were reconstituted in sterile cell culture grade water to 0.1 mg/ml, aliquoted, and stored at −20° C. Lipopolysaccharide (LPS, Enzo, ≥98% HPLC) from E. coli EH100 (Ra) was reconstituted in sterile cell culture grade water to 1.0 mg/ml and stored at 4° C.

To identify cytokine expression changes in chondrocytes treated with cannabidiol, or niacin alone or in combination, cells arrayed in 24-well cell culture plates were grown to 70% confluency, approximately-5.0×10⁴ cells per well, and then were treated for 24 hours with the test compounds in the absence of serum. Three wells were utilized per sample condition. Cytokines secreted over time by the cells under different treatments accumulate in the cell culture media, this is called conditioned media. Conditioned media was collected under sterile conditions, centrifuged at 3,000×g for 5 minutes at 4° C., and the supernatant was diluted to the appropriate volume. Lysates were taken for each sample to normalize resulting data based on cell number. Samples were shipped on dry ice to a third party for cytokine array analysis. Cytokine arrays provided by and analyzed by a third party company were used to detect distinct pro-inflammatory cytokines for each sample provided. Assays were performed using the Bio-Plex 200 system (BIO-RAD LABORATORIES). Assay sensitivities for each analyte/cytokine range from 0.11 to 3.25 picograms/milliliter. Raw data values were provided with corresponding standard curve values for each analyte.

A series of pilot studies were performed to design the final full-scale experiment. First, a toxicity screen for each test compound was performed to establishment maximum tolerable concentrations in this model system. Second, assays were run to establish the optimal means of inducing a pro-inflammatory state in the cell culture system. Third, conditions were tested to set the most favorable dilution scheme in order to maintain samples within the linear range of detection for each analyte. To establish the test concentrations for each test compound, chondrocytes were treated with a serial dilution of each compound, for 24 and 48 hours under serum free conditions. Oleocanthal or CBD were tested at two-fold dilutions down from 50 micromolar (μM). Niacin and Nicotinamide ribose at three-fold dilutions down from 1 mM. Cells were observed for signs of toxicity (vacuolation, blebbing, reduced cell count) by microscopy at 24 and 48 hours. The maximum tolerable concentration at 24 hours post-treatment for oleocanthal, CBD, niacin, and nicotinamide ribose were 604, 604, 1 mM and 1 mM respectively.

Experiments were then performed to optimize the model of a pro-inflammatory state. Chondrocytes were treated with a range of concentrations of IL-6, IL-1β, LPS, or combinations of each in order to stimulate the cells to produce pro-inflammatory cytokines. The cells were treated in the presence or absence of serum in the media, for 24 or 48 hours. Samples were collected for each of these conditions in varying combinations and shipped for third party analysis to detect the levels of different cytokines present in the conditioned media. From the results at least 8 of the 14 cytokines were detected in the array produced by the cells under optimal conditions. These optimal conditions were IL-10 stimulation alone, in absence of serum, for 24 hours. So for the data presented in the Figures the 24 hour treatments were: untreated; IL-1B alone; IL-1B plus the indicated compound(s).

For the purposes of the present invention, the levels “Low”, “Moderate”, and “High” relate to the dosage of the treatment of each replicate in the provided example, in accordance with Table 1, below correlating with the data set forth in the Figures regarding the exemplary embodiment.

TABLE 1
Concentration/Dosage levels for Low, Moderate, and High concentrations.

| | Final | IL1β | Oleocanthal | Cannabidiol | Niacin | Nicotinamide |
|---|---|---|---|---|---|---|
| | DMSO | (ng/ml) | (μM) | (μM) | (μM) | Ribose (μM) |
| Untreated | <.1% | 10 | | | | |
| IL1β | <.1% | 10 | | | | |
| CBD low | <.1% | 10 | | 0.67 | | |
| CBD moderate | <.1% | 10 | | 2.00 | | |
| CBD high | <.1% | 10 | | 6.00 | | |
| Niacin low | <.1% | 10 | | | 100.00 | |
| Niacin moderate | <.1% | 10 | | | 300.00 | |
| Niacin high | <.1% | 10 | | | 900.00 | |
| Niacin/CBD low | <.1% | 10 | | 0.67 | 100.00 | |
| Niacin/CBD moderate | <.1% | 10 | | 2.00 | 300.00 | |
| Niacin/CBD high | <.1% | 10 | | 6.00 | 900.00 | |
| Niacin/OC/CBD low | <.1% | 10 | 0.67 | 0.67 | 100.00 | |
| Niacin/OC/CBD | <.1% | 10 | 2.00 | 2.00 | 300.00 | |
| moderate | | | | | | |
| Niacin/OC/CBD high | <.1% | 10 | 6.00 | 6.00 | 900.00 | |
| NR low | <.1% | 10 | | | | 100.00 |
| NR moderate | <.1% | 10 | | | | 300.00 |
| NR high | <.1% | 10 | | | | 900.00 |

To ensure the maximum amount of information possible from the present example, a second pilot assay was performed comparing standard and high sensitivity arrays at a series of sample dilutions. This was necessary to ensure analytes were within the optimal linear detection range. It was determined that two-fold dilutions of each sample should be assayed on the high-sensitivity arrays.

The results of the present example are set forth in the Figures. From the above described example there are multiple principle findings that confirm the tested compounds act additively or synergistic in combination to repress production of pro-inflammatory cytokines by chondrocytes.

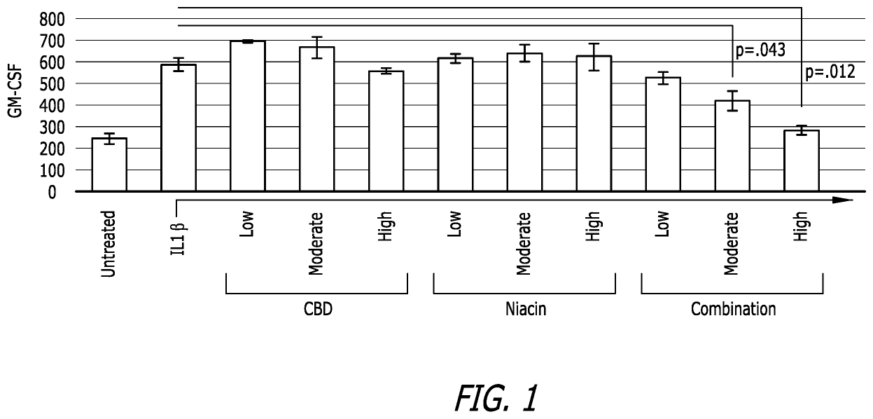

Turning to the Figures, FIG. 1. depicts a chart showing the synergistic effect of combination therapies in relation to non-combination therapies on expression of GM-CSF by chondrocytes. As known by one of skill in the art, GM-CSF is a glycoprotein cytokine that has a priming effect on IL-13 mRNA and protein expression. Results shown include administration of various levels of CBD or Niacin, either alone or in combination, including Low, Moderate, and High levels of administration, as further defined herein. From the results, the administration of Moderate and High levels of the combination of CBD and niacin is observed to synergistically reduce the expression of GM-CSF in a significant manner. This effect was not seen with either compound alone.

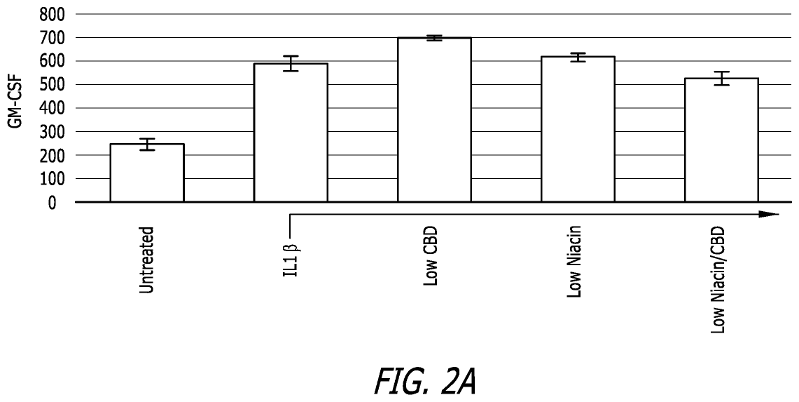

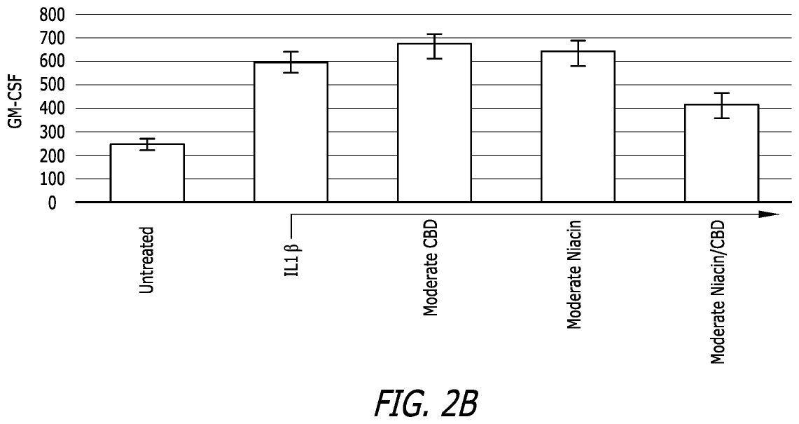

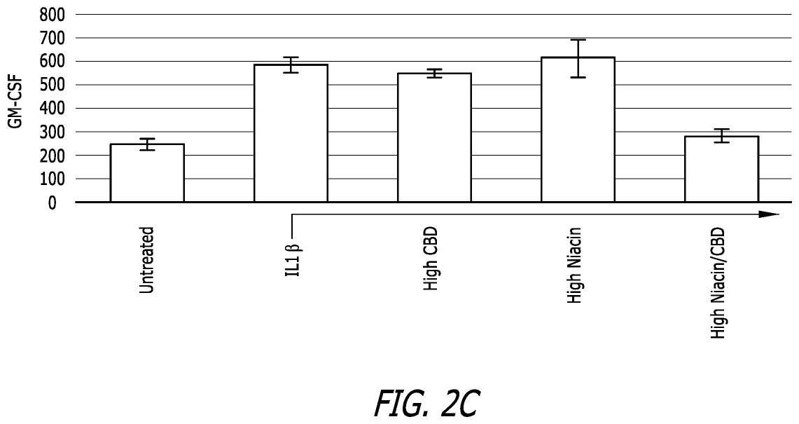

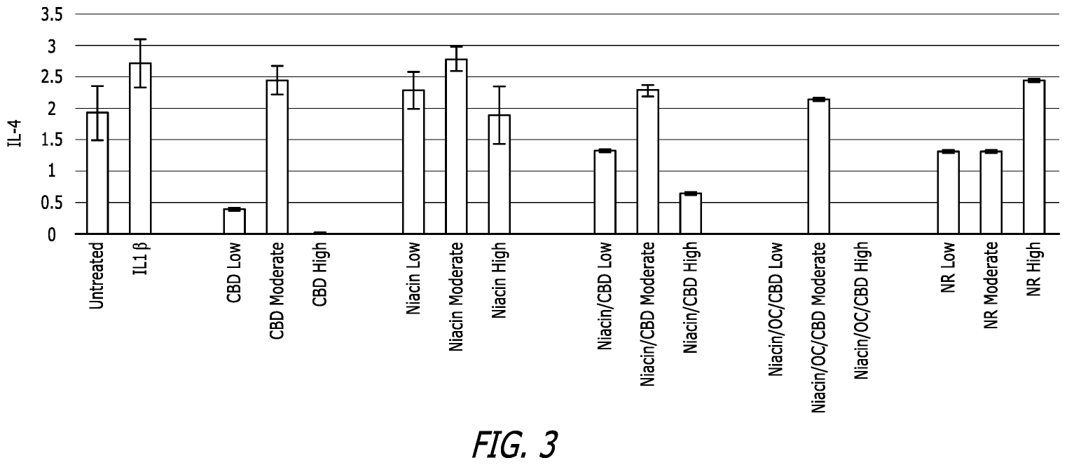

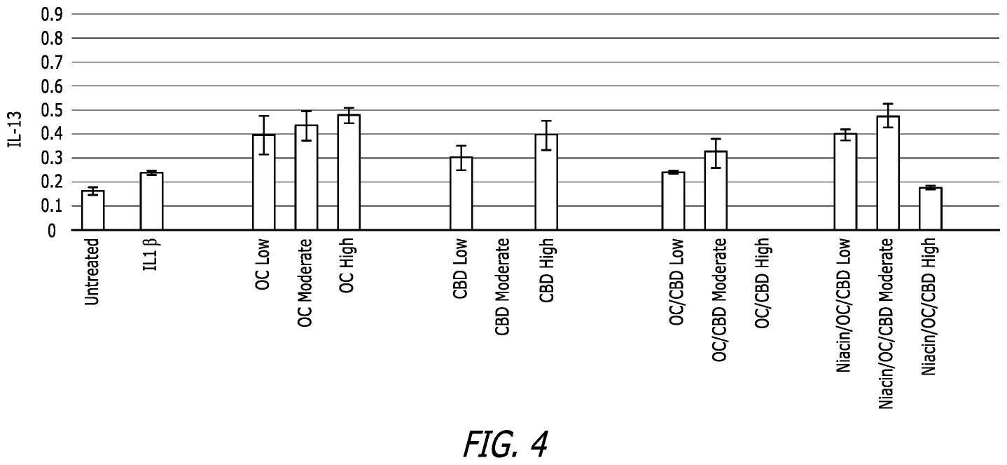

FIG. 2A further presents data showing Low concentrations of individual and combination therapies and the corresponding effect on GM-CSF expression. From the data presented in FIG. 2A, the Low concentration of the combined dosage of CBD and Niacin shows limited anti-inflammatory effects. FIG. 2B presents data showing Moderate concentrations of individual and combination therapies and the corresponding effect on GM-CSF expression. From these results, the combination dose in a Moderate dosage is shown to have improved anti-inflammatory effects. FIG. 2C presents data showing High concentrations of individual and combination therapies and the corresponding effect on GM-CSF expression. From the results it is made clear that the High dose combination treatment shows significantly improved anti-inflammatory effects. FIG. 3 depicts a chart showing IL-4 expression as a result of varying concentrations of individual and combination therapies (Low, Moderate, and High). From the data provided, it is shown that Low, Moderate and High levels of the combination treatments show anti-inflammatory effects. FIG. 4 presents data showing IL-13 expression as a result of varying concentrations of individual and combination therapies. From the results, High combination treatments show increased anti-inflammatory effects.

From the above described example there are multiple principle findings that confirm the tested compounds act additively or synergistic in combination to repress production of pro-inflammatory cytokines by chondrocytes. In one aspect, Moderate and High concentrations of CBD and niacin act synergistically in combination to repress GM-CSF production (see FIGS. 2A-C). It is believed that the results are applicable to treatment of all animals including humans, pet animals, zoo animals, livestock and farm stock animals. Preferably each dosage of a formulation according to the present disclosure provides from 0.1 to 100 milligrams of the at least one cannabinoid in each dosage.

It is noted that the foregoing examples have been provided merely for the purpose of explanation and are in no way to be construed as limiting of the present invention. While the present invention has been described with reference to exemplary embodiments, it is understood that the words, which have been used herein, are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

### REFERENCES

1. Antiseizure properties of cannabidiol (CBD) are attenuated in the absence of transient receptor potential vanilloid 1 (TRPV1) receptors (S53.004) Benjamin J. Whalley, Royston A. Gray, Colin G. Stott, Nicholas A. Jones Neurology April 2018, 90 (15 Supplement) 553.004.
2. Costa B l, Trovato A E, Comelli F, Giagnoni G, Colleoni M. The non-psychoactive cannabis constituent cannabidiol is an orally effective therapeutic agent in rat chronic inflammatory and neuropathic pain. Eur J Pharmacol. 2007 Feb. 5; 556(1-3):75-83. Epub 2006 Nov. 10.

1. A composition for treatment of inflammatory conditions in an animal, said composition comprising:
at least one cannabinoid; and
at least one NAD+ precursor;
wherein said composition is capable of having an anti-inflammatory effect when administered to an animal.

2. The composition of claim 1, wherein the molar ratio of the at least one cannabinoid and the at least one NAD+ precursor is between 200:1 and 1:200.

3. The composition of claim 1, wherein the molar ratio of the at least one cannabinoid and the at least one NAD+ precursor is between 150:1 and 1:150.

4. The composition of claim 1, wherein the molar ratio of the at least one cannabinoid and the at least one NAD+ precursor is 100:1 to 1:100.

5. The composition of claim 1, wherein said composition is suitable for administration selected from a group consisting of: oral administration, topical administration, mucosal administration, pulmonary administration, subcutaneous administration, intravenous administration, intraperitoneal administration, suppository administration, and intramuscular administration.

6-9. (canceled)

10. The composition of claim 1, wherein said composition is a formulation selected from the group consisting of: a tablet, capsule, spray, drop, solution, suspension, gel, ointment, lotion, cream, powder, transdermal patch, tampon, or a sponge.

11. The composition of claim 1, wherein the at least one NAD+ precursor comprises nicotinic acid.

12. The composition of claim 1, wherein the at least one NAD+ precursor is selected from the group consisting of: tryptophan, nicotinic acid, nicotinamide, nicotinamide riboside, nicotinamide ribose, nicotinamide mononucleotide, and combinations thereof.

13. The composition of claim 1, wherein the cannabinoid is cannabidiol (CBD).

14. The composition of claim 1, wherein the cannabinoid is selected from the group consisting of: cannabidiol (CBD), cannabidivarol (CBDV), cannabinol (CBN), cannabigerol (CBG), cannabivarol (CBV), cannabicyclol (CBL), tetrahydrocannabinol (THC), tetrahydrocannabinol-C4, (THC-C4), tetrahydrocannabivarin (THCV), 11-Hydroxy-Δ9-tetrahydrocannabinol, (11-OH-THC), 11-nor-9-Carboxy-Δ9-tetrahydrocannabinol, and combinations thereof.

15-28. (canceled)

29. A method for preparing a combination formulation having anti-inflammatory properties, the method comprising the steps of:
providing at least one cannabinoid;
providing at least one NAD+ precursor; and
combining the at least one cannabinoid with the at least one NAD+ precursor to form a combination formulation;
wherein said combination formulation is capable of reducing inflammation in an animal administered said combination formulation.

30. The method of claim 29, wherein the molar ratio of the at least one cannabinoid and the at least one NAD+ precursor in the combination formulation is between 200:1 and 1:200.

31. The method of claim 29, wherein the molar ratio of the at least one cannabinoid and the at least one NAD+ precursor in the combination formulation is between 150:1 and 1:150.

32. The method of claim 29, wherein the molar ratio of the at least one cannabinoid and the at least one NAD+ precursor in the combination formulation is 100:1 to 1:100.

33. The method of claim 29, wherein said combination formulation is suitable for administration selected from a group consisting of: oral administration, topical administration, mucosal administration, pulmonary administration, subcutaneous administration, intravenous administration, intraperitoneal administration, suppository administration, and intramuscular administration.

34.-37. (canceled)

38. The method of claim 29, wherein said combination formulation is a formulation selected from the group consisting of: a tablet, capsule, spray, drop, solution, suspension, gel, ointment, lotion, cream, powder, transdermal patch, tampon, or a sponge.

39. The method of claim 29, wherein the at least one NAD+ precursor comprises nicotinic acid.

40. The method of claim 29, wherein the at least one NAD+ precursor is selected from the group consisting of: tryptophan, nicotinic acid, nicotinamide, nicotinamide riboside, nicotinamide ribose, nicotinamide mononucleotide, and combinations thereof.

41. The method of claim 29, wherein the cannabinoid is cannabidiol (CBD).

42. The method of claim 29, wherein the cannabinoid is selected from the group consisting of: cannabidiol (CBD), cannabidivarol (CBDV), cannabinol (CBN), cannabigerol (CBG), cannabivarol (CBV), cannabicyclol (CBL), tetrahydrocannabinol (THC), tetrahydrocannabinol-C4, (THC-C4), tetrahydrocannabivarin (THCV), 11-Hydroxy-Δ9-tetrahydrocannabinol, (11-OH-THC), 11-nor-9-Carboxy-Δ9-tetrahydrocannabinol, and combinations thereof.
